(19) United States
(12) Patent Application Publication
(10) Pub. No.: US 2004/0078063 A1
(43) Pub. Date: Apr. 22, 2004

(54) Electrical apparatus and methods for denaturing venoms and toxins

(76) Inventor: Brian McLaren, Oklahoma City, OK (US)

Correspondence Address: Dunlap, Codding & Rogers, P.C., P. O. Box 16370, Oklahoma City, OK, 73113, US

(21) Appl. No.: 10/277,391

(22) Filed: Oct. 17, 2002

(51) Int. Cl. A61N 1/18

(52) U.S. Cl. 607/46

(57) ABSTRACT

An apparatus and method for electrically denaturing venoms and toxins having a housing, a voltage generator, and a distributor system with a terminal and a plurality of electrodes.

### CROSS REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

In nature venoms serve a number of functions. They may be injected through a bite or sting and act to immobilize a prey, or assist in the pre-digestion of such prey once caught. Venoms may also be exuded through the skin to provide a protective function for that particular individual. Understanding the common nature of all venoms allows an advanced method to be developed to neutralize the tissue effects of both neurotoxic and cytolytic venoms.

Humans are exposed to several different venomous species. Envenomation by such venomous species can cause minor injuries, serious injuries, or even death if exposure to certain venoms is not treated or if an individual has an allergic reaction. Examples of such species include spiders (i.e., Brown Recluse or Black Widow), snakes (i.e., Rattle snakes, Water Mocassins, or Copperheads), bees, wasps, yellow jackets, hornets, ants, scorpions, venomous lizards, ticks, mites, centipedes, millipedes, marine coelenterates (i.e., Portuguese man-of-war, Sea wasp, or Box jellyfish), and the like.

Venoms are extremely complex physiological mixtures that may be described as proteinacious fluids with enzymatic activity, plus an accompanying array of smaller polypeptide moieties that are, in certain cases, the toxic component. A protein's structure is the result of a delicate balance among powerful counter-veiling electrostatic forces (non-covalent, covalent, polar and ionic), yet thermodynamic measurements indicate that native proteins are only marginally stable entities under physiological conditions.

Underlying the skin in the lower dermis and subcutaneous tissue are the majority of all sensory nerves. By damaging the skin and subcutaneous tissue, information is relayed to the Central Nervous System (CNS), which consists of the brain and the spinal cord. At the level of the spinal cord, within hours of peripheral damage, there is a change in the expression of the DNA and the neuro-chemicals and modulators released.

When the skin and subcutaneous tissue is traumatized and venom deposited, the change in the electrical field is transduced by collagen to stimulate nerves to transmit a message such as pain to the brain electrically. Pain may be shown to not only be transmitted by the release of primary messengers such as prostaglandin and other eicosanoids, which are released from damaged cells, but pain can also be shown to be due to a lowering of the oxygen tension in the tissue (for example, appling a tourniquet does not damage tissue but can cause severe pain). Another way in which pain is created is by lowering the pH of the interstitial fluids from normal (Ph of about 7.4) to low (pH of about 6.1).

As the cell changes electrically and chemically, it is also changing the relative acidity of the inside to the outside of the cell. This is important as a cell can only operate within a very narrow pH range, and extremely small changes can affect a cell tremendously.

Neurons, like other cells, generate ionic gradients across their plasma membranes through the actions of the corresponding ion-specific pumps. In particular, potassium (K) is pumped into and Sodium (Na) is pumped out of the nerve cell to yield intracellular and extracellular concentrations across the cell membrane. The membrane potential is generated by a surprisingly small imbalance in the ionic distribution across the membrane. Only approximately one ion pair per million are actually separated by the membrane, with the anion going to the inner cytoplasmic side, and the cation going to the external side. The resulting electric field is, never the less, enormous by macroscopic standards, being nearly 170,000 Volts per centimeter.

All cells generate ionic gradients across their plasma membranes through the action of the corresponding ion-specific pumps. An action potential is triggered by approximately a 20 m-Volt rise in membrane potential, which at rest is approximately 90 mVolts. The ion specific permeability changes that characterize an action potential result from the presence of trans-axonal membrane proteins that function as either sodium or potassium specific voltage-gated channels.

In experimentally measured membrane potential for mammalian cells, the quantities and the respective permeability coefficients for the various cations and anions are indicative of how readily the corresponding ions traverse the membrane, each being equal to the corresponding ions diffusion coefficient through the membrane divided by the membrane's thickness. The membrane potential is generated by a surprisingly small imbalance in the ionic distribution across the membrane.

The changes in the ion-specific permeability of the cell membrane result from the presence of transmembrane proteins that function as Na⁺, K⁺, or Ca⁺² specific voltage-gated channels. Many neurotoxins interfere with the voltage-gated Na⁺ channels, while others affect the K⁺ or Ca⁺² channels only from the outside of the cell. Neurotoxins affect the various mechanistic aspects of neuro-transmission. Many neurotoxins interfere with the action of voltage-gated sodium channels but curiously fewer are known that affect potassium channels.

Many neurotoxins have a cationic guanidino group that is effective only when applied to the external surface of a neuron, while their injection into the cytoplasm elicits no response. It is therefore thought that these toxins specifically interact with an ionic carboxylate group located at the mouth of the sodium channels on the extracellular surface, blocking the sodium channel.

A steroid alkaloid secreted by the skin of a Colombian arrow-poison frog is the most potent known venom. Two micrograms per kilogram body weight is lethal. This substance also binds to voltage-gated sodium channels, but in contrast to the actions of other neurotoxins renders the axonal membrane highly permeable. Many toxins do not compete with each other for binding to the sodium channels and therefore must bind at separate sites.

The mammalian nervous system employs well over thirty substances as neuro-transmitters; most of these substances are amino acids (the building blocks of protein), or their breakdown derivatives. All venoms are toxins, which consist of proteins either singly or in multiple arrays. In the case of the Southwestern American Scorpion (Centruoides scuipturcaus) there are “families” of sixty to seventy residue-protein neurotoxins that act to depolarize neurons by binding to their Na⁺ (Sodium ion) channels. The different neurotoxins in the same venom appear to be specialized for binding to the Na⁺ channels in the various species the scorpion is likely to encounter in the desert. Thus, when a scorpion stings the human gets the entire load of different toxins, and the venom is not easily detoxified chemically. This accounts for the difficulty in producing an antivenom serum.

As previously stated, many neurotoxins interfere with the voltag-egated Na⁺ channels. For example, the arrival of an action potential at the neuromuscular junction triggers the opening of voltage-gated Calcium (Ca⁺²) channels. The resulting influx of extra cellular Ca⁺² into the nerve terminal in-turn stimulates the release of chemicals (such as acetylcholine) which stimulate muscle contraction. The Black Widow spider takes advantage of this system; its highly neurotoxic venom protein causes massive release of acetyleholine at the neuromuscular junction, causing massive muscular contractions. This would prevent a person from relaxing and breathing out. This system, where death occurs through respiratory arrest, is the target of some of the most deadly known neurotoxins, for example, a family of homologous venom proteins from some of the world's most poisonous snakes, including certain sea-snakes and cobras.

In contrast, the chemical release discussed above is inhibited by botulinum toxin (a mixture of eight proteins produced by the bacteria Clostridium botulinum), the agent responsible for the deadly food poisoning, botulism.

All enzymes are proteins, and they differ from ordinary chemical catalysts in that:

they have higher reaction rates (up to 100 million times greater than chemically catalyzed reactions),

they operate through a larger range of environmental conditions,

they often show a greater degree of specificity with regard to their substrates,

they produce virtually no side products compared to chemical catalysts,

most importantly, their catalytic activity varies in response to the concentration of substances other than their specific substrate.

Most proteins function through their shape, and this is known as allosteric interactions. One of the main mechanisms of control of an enzyme's activity is through the alteration of its conformational or structural detail. Proteins have lifetimes that range from as short as a few minutes to many months. Within a cell proteins are constantly being synthesized from and degrading back into their component amino acids. The function of this seemingly wasteful process is twofold, in that it eliminates abnormal proteins whose accumulation would be harmful to the cell, and it permits the regulation of cellular metabolism by eliminating superfluous enzymes and regulatory proteins.

Venoms may contain a number of different enzymes, each of which can have a range of functions. The enormous complexity of cellular function tends to make it vulnerable in a number of ways.

It is common to hear quite gruesome stories of necrotizing wounds and severe pain that lasted many months following certain spider bites. Current medical treatment often includes surgical excision, debridement, plus a skin graft over an area of 1½ inch diameter, with the resulting area discolored and still very sore twelve to eighteen months later.

The Black Widow Latrodectus mactans (and related species, with neurotoxic venom) and the Brown Recluse Loxosceles reclusa (also known as Violin or Fiddle-back, with necrotizing venom) are the two most common native venomous spiders in the USA. While Tarantulas and Banana Spiders exist, they are not native to the USA.

Spiders such as the Brown Recluse are small and hunt their prey by injecting a substance that will predigest and liquefy their prospective meal. To cause this action, the venom contains an enzyme that disrupts the integrity of the cellular function of the host's tissues. This may be directly through the action on the cell wall itself, or indirectly in that the enzyme may either prevent normal cellular degradation of abnormal (and potentially damaging) proteins, or by indiscriminately increasing the normal protein breakdown function in a cell.

Venomous stinging insects include bees, wasps, yellow jackets, hornets, and ant. In the United States, stings from such insects are responsible for three to four times more deaths per year than are venomous snake bites. Fire ant venom has hemolytic, cytolytic, antimicrobial and insecticidal properties, and only three or four small aqueous protein fractions are responsible for the allergic reaction. Scorpions have also been known to inflict many injuries that have resulted in deaths in children under six years as well as hypersensitive patients. In addition, other biting arthropods such as ticks and mites are not considered venomous but may transmit diseases that have been responsible for fatalities in humans. While centipedes can cause severe bites, they are not dangerous but millipedes secrete a toxin through their skin that causes local irritation and in severe cases vesiculation and necrosis. Marine Coelenterates (Portuguese man-of-war, Sea wasp, Box jellyfish) are responsible for more envenomations than any other marine animal. Of the 9,000 species, 100 are toxic to humans.

There are a number of gaps in the existing knowledge:

The absolute current to amperage ratio (total dose), and also the applied dose rate (dose/unit of time) of direct current (DC) to neutralize venom is unknown.

Most venom components appear to bind with multiple physiological receptor sites in a victim, therefore the arbitrary classification into neurotoxins, hemotoxins, necrotizing, cytolytic or cardiotoxins are superficial and can lead to serious errors in clinical judgment.

Conventional methods of treating either snakebite or spider bite has as a sequel, serum sickness in 75% of treated patients, within seven to twenty-one days that requires further treatment.

It is unknown what would be the effect of using the present crude electrical treatments on someone who had been given anti-venom.

It is unknown how long after having been bitten or stung would electrical treatment still be the modality of first choice.

It is unknown what is a safe level of electrical shock that could be given to a child, an epileptic person or someone with a pacemaker.

It is unknown if the electric current applied to a bite on the neck or limb can be isolated from the head or body.

There are also faults with electrical equipment presently available with which to treat envenomation. While it is reasonably easy to test below 15,000 Volts or above the mega range, there are idiosyncrasies of test equipment for the 100,000 Volt range. It is believed that this is one reason why existing 100,000 Volt stun-guns have been modified without being quantified. Alternating current (AC) electrical equipment already exists in a non-commercial, clinically unacceptable form. It is well known that a direct current (DC) is some three to five times less dangerous than an alternative current (AC), of the same value, yet the method of delivery at present is AC. While it is accepted that low frequency pulse rates, e.g., 60 Hz are more dangerous than higher frequencies, the present generation of AC stun guns are all low frequency pulsed. The AC (or pulsating DC) equipment presently available, works on the principle that the electric current has to be strong enough to flow through the limb to the ground outlet. However, it is known that dermatological and musculoskeletal injuries are associated with significant changes in the endogenous electrical activity in the vicinity of the injury, and that this electrical activity is directly associated with the injured epidermis (the subcutaneous connective tissue is comprised of collagen, a protein, which is piezoelectric).

Bearing in mind that each electrical charge produces an electric field in its vicinity, with the total electric field due to one or more charges being the sum of their individual electrical fields. If the electric fields point in different direction, cancellations will occur in the sum (these cancellations may cause the total field to vary with distance, in a way that is quite different from the inverse distance squared dependence of the individual point charge fields).

It is standard knowledge that if an electrically charged particle Q, is brought into contact with an existing field E, it experiences a force proportional to the electric field and the charge itself, where F=QE.

Due to the intricacy and sophistication of the multiple actions of venom the possibility of finding a single chemical or biological antidote, that will not harm the host tissue, is often remote.

By utilizing existing knowledge of skin impedance, tissue conductance, and the energy required to break Hydrogen bonds (which after all are only electrostatic reactions), a safer method of denaturing venoms (allosteric proteins, stereo-specific enzymes, and polypeptide moieties) has been developed.

A type of secondary results that has been anticipated is that venoms from different phylum, or class (e.g., snakes, spiders, marine coelenterates) may require a different electrical charge to denature them. If the charge difference was significant then a modification to the unit could be built to allow a person to dial in the required “Spider” or “Snake” dose.

Therefore, rather than electrocuting the whole limb, subtle changes at a local level are all that is required to reduce the pain, regulate the bodies immune response (which in the case of anaphylaxis is an exaggerated response that may be life threatening), detoxify the venom, and promote rapid healing.

It is known that an electrical charge may neutralize some venom but because the mode of action is not well understood, the function of the electrical charge in tissue is often misrepresented. It is proposed to biometrically quantify the absolute current to amperage ratio (total dose), and also the applied dose rate (dose/unit of time) of direct current (DC) that is necessary to allosterically alter the proteinacious nature of the venom, without damaging the tissue.

There are alternating current (AC) stun guns (reputedly of at least 100,000 Volts) that some medical practitioners have crudely modified to function as an electro-antivenene device. For instance, a modified AC stun gun has been utilized in which an alligator clip with a small lead has been attached to one probe while the other end of the wire was used as the ground on some part of the patient's body. The problems with this method are:

It is well known that a direct current (DC) is some three to five times less dangerous than an alternative current (AC), which is presently the method of delivering the electric shock,

Uncontrolled distance from the skin (variable level of shock to the patient), due to the level of charge required to jump the intervening distance,

An inability to accurately direct the spark to a particular point (The doctor has to “paint” the wound),

Variations in the distance the current has to travel through or around the limb or area, plus

The fact that some of the time the doctor also receives an electric shock.

Therefore, there exists a need in the art for new and improved apparatus and methods for treatment of venomous bites and stings that overcome the disadvantages of the prior art. It is to such new and improved apparatus and methods that the present invention is directed.

### BRIEF DESCRIPTION OF THE DRAWINGS

FIG. 1 is a perspective view of an apparatus for denaturing venoms and toxins disposed over an area of envenomation on a patient's hand.

FIG. 2 is a partial cut away view of the housing of the apparatus of FIG. 1 showing a voltage generator and a distribution system of the apparatus.

FIG. 3 is a side elevational view of the housing of the apparatus of FIG. 1.

FIG. 4 is a schematic diagram of the voltage generator of the apparatus of FIG. 1.

FIG. 5 is a side cut away view of the apparatus of FIG. 1 showing the distributor system of the apparatus in more detail.

FIG. 6 is a front elevational view of the housing showing a terminal and a plurality of electrodes of the distributor system of FIG. 5.

### DETAILED DESCRIPTION OF THE INVENTION

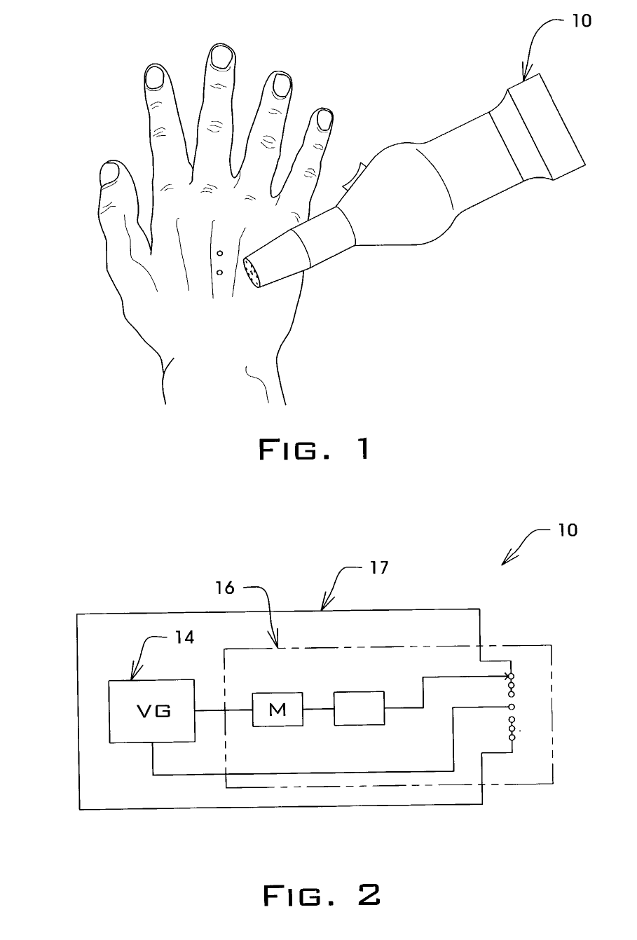

Referring now to the drawings and, more particularly to FIG. 1 shown therein, is an apparatus 10 for denaturing venoms and toxins constructed in accordance with the present invention positioned over an area of envenomation on the hand of a patient. The apparatus 10 for denaturing venoms and toxins includes a voltage generator 14, a distributor system 16 and a housing 17 (FIG. 2).

The voltage generator 14 generates an appropriate voltage and amperage level needed to denature venoms and toxins. The distributor system 16 receives and disperses the appropriate voltage and amperage levels generated by the voltage generator 14 over the area of envenomation. The housing 17 substantially encloses and supports the voltage generator 14 and the distributor system 16.

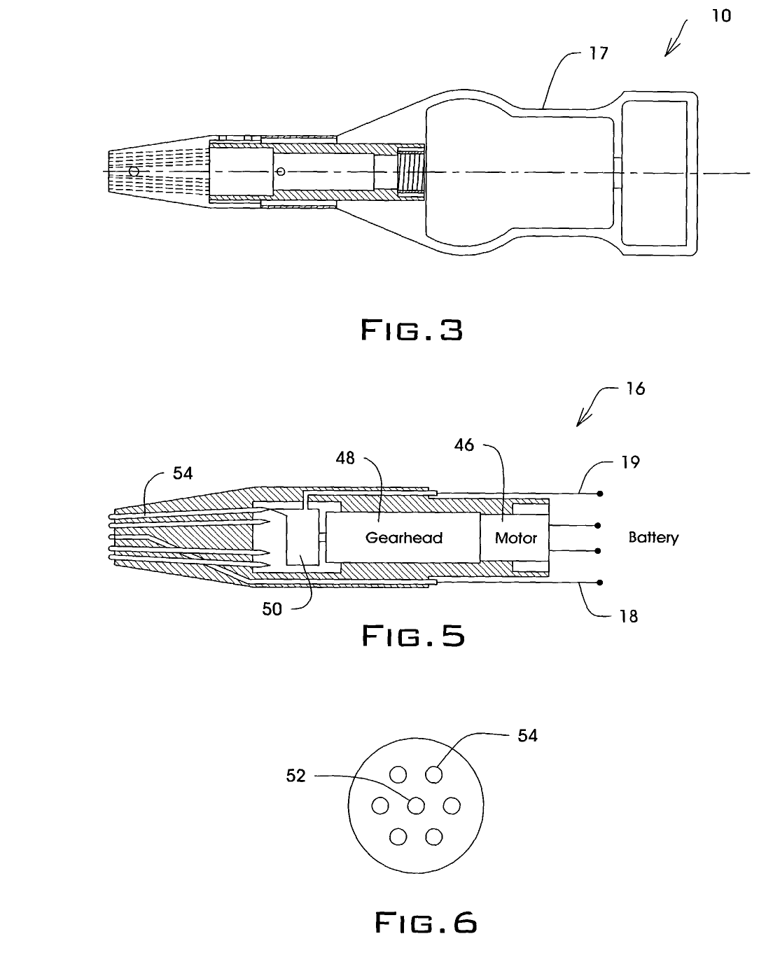

Referring to FIG. 3, shown therein is the housing 17 for the apparatus 10. The housing 17 can be any convenient size or shape. The housing 17 is substantially constructed from non-conducting material such as plastic, fiberglass, wood or combinations and derivations thereof. The housing 17 facilitates grasping of the apparatus 10 by the operator and placement of the apparatus 10 on the patient. The housing 17 at least partially encloses and supports the voltage generator 14 and the distributor system 16 so as to protect the operator from receiving an accidental shock from the apparatus 10 and yet sufficiently exposes a plurality of electrodes and a terminal (to be described hereinafter) such that the terminal and the plurality of electrodes can easily and conveniently be disposed on a patient.

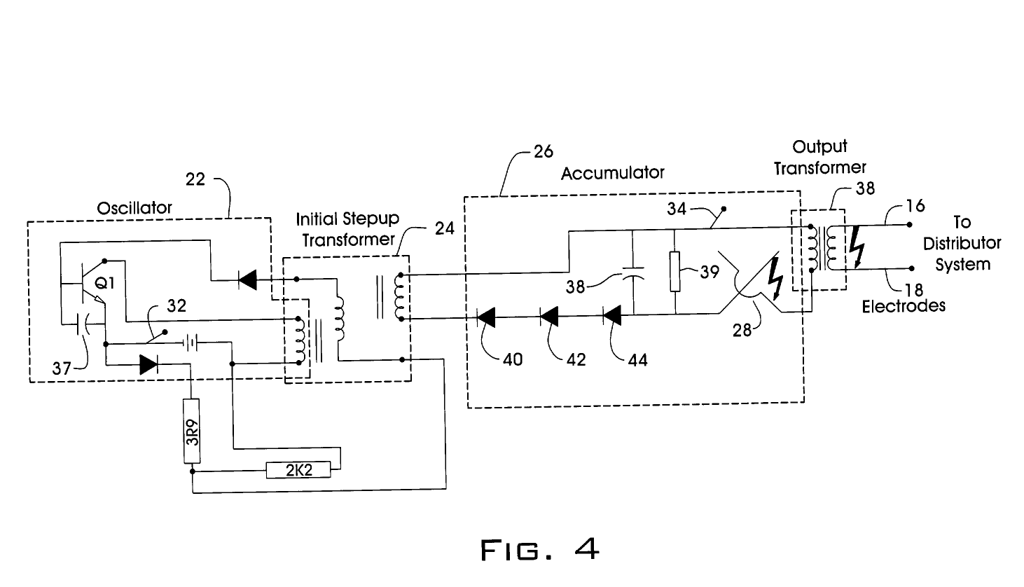

Referring now to FIG. 4, typically, the voltage generator 14 generates an output voltage between a first output 18 of the voltage generator 14 and a second output 19 of the voltage generator 14 in an approximate range from about 15 kilovolts to about 22 kilovolts at amperage levels in a range generally from about 13 milliamps to about 18 milliamps. The voltage and amperage levels within these approximate ranges denature the venom and toxins. It should be noted that higher voltage and amperage levels can be utilized to denature the venoms and toxins, however, excessive voltage and amperage levels can cause unnecessary discomfort or even harm to the patient. It should also be noted that lower voltage and amperage levels can be utilized to denature the venoms and toxins, however, inadequate voltage and amperage levels may not sufficiently denature the venoms and toxins or only denature a small area of envenomation. The voltage generator 14 can be a set voltage generator that generates a predetermined output voltage within the approximate range from about 15 kilovolts to about 22 kilovolts or the voltage generator 14 can be a variable voltage generator for generating a variable output voltage level that can be varied, at the discretion of the operator, preferably within the approximate range from about 15 kilovolts to about 22 kilovolts. The advantage of the variable voltage generator is that the voltage level, or in other words the dosage amount given to the patient can be controlled by the operator.

In one embodiment the voltage generator 14 of the apparatus 10 includes the first output 18 and the second output 19, an oscillator 22, an initial stepup transformer 24, an accumulator 26, an arc gap switch 28, an output transformer 30, a first switch 32, a second switch 39 and a battery 36. The first switch 32 turns on the apparatus 10, charges the voltage generator 14 and turns off the apparatus 10 thereby disabling the apparatus 10. The second switch 34 activates the distributor system 16 and discharges the apparatus 10 after the apparatus 10 has been charged by turning the first switch 32 to the on position.

The oscillator 22 steps up the voltage supplied by the battery 36. The oscillator 22 is preferably a Hartley Oscillator. The oscillator 22 oscillates at the natural frequency of the primary transformer windings of the initial stepup transformer 24 and a capacitor 37 thereby generating a time varying signal. The voltage level of the time varying signal is increased by the initial stepup transformer 24 and the output transformer 30 of the voltage generator 14.

One advantage of the Hartley Oscillator over other oscillator circuits, is that the current which is used to excite the primary windings of the initial stepup transformer 24 immediately induces a current in the secondary windings of the initial stepup transformer 24 without need of additional impedance matching. The oscillator 22, therefore, uses the initial stepup transformer 24 as both a source of the oscillation and as a means for stepping up the voltage level of the time varying signal. Although the high voltage generator 14 is shown as including the oscillator 22, any discrete or integrated circuit which produces a time varying signal capable of being stepped up can be utilized. Furthermore, although the time varying signal is shown as being stepped up by the initial stepup transformer and output transformer, the time varying signal can be stepped up by a voltage multiplier and a power amplifier or even a voltage multiplier, power amplifier and stepup transformer all in combination. Voltage multipliers and power amplifiers and setpup transformers are well known in the art and no further description is deemed necessary.

The initial stepup transformer 24 affects the frequency of the time varying signal and steps up the voltage level of the time varying signal simultaneously. The initial stepup transformer 24 also provides the additional benefit of isolating the relatively low voltage components of the oscillator 22 from the high voltage components of the voltage generator 14.

The secondary winding of the initial stepup transformer 24 forms a loop with a high voltage capacitor 38 and a resistor 39. The oscillator 22 continues to feed power through the initial stepup transformer 24. The current induced in the secondary windings is circulating in a capacitor loop and the high voltage capacitor 38 charges up until discharged by the arc gap switch 28. First, second and third diodes 40, 42 and 44, also present in this loop, rectify the current so that it travels in one direction (ignoring the diodes small reverse current), causing a direct current charge to be accumulated across the high voltage capacitor 38. The first, second and third diodes 40, 42 and 44 are used in series to increase the overall breakdown voltage rating of the first, second and third diodes 40, 42 and 44 in combination.

The arc gap switch 28 basically acts as a timing mechanism to allow the accumulator 26 enough time to build up sufficient power to excite the output transformer 30. The arc gap switch 28 also delivers a very fast pulse of energy to the output transformer 30. The arc gap switch 28 consists of two metal plates or wires which are typically separated by approximately half a millimeter of an air dielectric. If the distance between the two metal plates of the arc gap switch 28 is too large, the frequency of the arc gap discharge is decreased and the circuit cannot accumulate enough voltage to jump the arc gap. This condition could damage the high voltage capacitor 38 in the circuit because of the voltages accumulated and the inability of the voltages to be discharged. If the distance between the two metal plates is too small, excessive current and excess heat are generated and there is insufficient power to excite the output transformer 30. Once sufficient voltage is accumulated by the high voltage capacitor 38, an electric discharge is generated across the arc gap switch 28. The electric discharge across the arc gap switch 28 discharges all of the energy stored up in the high voltage capacitor 38.

Preferably the output transformer 30 is a toroidal transformer with a ferrite core. This type of transformer, allows the output transformer 30 to have a high inductance and the ability to utilize a large amount of electrical energy before magnetic saturation.

In operation, the arc gap switch 28 causes a large, fast flowing current through a primary of the output transformer 30. This large fast flowing current through the primary is transformed to the output voltage in the range of about 15 kV to 22 k. The 15 kv to 22 kv output voltage does have a small component which appears negative, however, this small negative component is unavoidable and is due to the electrical inertia of the circuit. Although the high voltage generator 14 is shown and described basically as the oscillator 22, the arc gap switch 28 and initial stepup transformer 24 and output transformer 30 those skilled in the art will readily recognize and appreciate that there are a wide variety of ways within which to produce the voltage and amperage levels within the preferred voltage and amperage levels described herein.

Referring now to FIG. 5, the distributor system 16 includes a motor 46, a gear box 48, a rotor 50, a terminal 52 and a plurality of electrodes 54. Each one of the plurality of electrodes 47 is electrically isolated from all of the other electrodes of the plurality of electrodes 54. The motor 46 is powered by the battery 36. The motor 46 is activated by the second switch 34. The gear box 40 is connected to the motor 46 and the rotor 50 such that as the motor 46 is actuated by the second switch 34, the rotor 50 turns gears within the gear box 48 which in turn spins the rotor 50. The rotor 50 is conductively connected with the second output 19 of the high voltage generator 14. The rotor 50 intermittently makes contact with each of the plurality of electrodes 54 as the motor 46 turns the gears within the gear box 40 which spins the rotor 50 and thereby distributes the voltage and amperage generated by the voltage generator 14 between the terminal 52 and the plurality of electrodes 54 so as to electrically denature the venoms and toxins near the area of envenomation on the patient. The distributor system 16 connects the second output 19 of the voltage generator 14 to each one of the plurality of electrodes 54 in proximity to the terminal 18, such that, the voltage potential generated by the voltage generator 14 is discharged between the terminal 52 of the voltage generator 14 and each of the plurality of electrodes 54.

Referring now to FIG. 6, typically the plurality of electrodes 54 surround the terminal 52 of the distributor system 16 in a circular pattern. Those skilled in the art, however, will recognize and appreciate that plurality of electrodes 54 can also surround the terminal 52 in a hexagonal pattern, an octagonal pattern or combinations and/or derivations thereof substantially equal in distance from the center of the electrodes.

In use, the operator turns the apparatus 10 on utilizing the first switch 32. The operator grasps the housing 17 and the plurality of electrodes 54 and the terminal 52 are placed in contact with the area of envenomation on a patient. The motor 46 is energized by the battery 36 via the second switch 34. The motor 46 rotates the gears within the gear box 48 which in turn rotates the rotor 50 which makes electrical contact with each of the plurality of electrodes 54, the output voltage generated by the voltage generator 14 is discharged between the terminal 52 and each of the plurality of electrodes 54 thereby dispersing the output voltage so as to electrically denature venoms and toxins at or near the site of envenomation on the patient. Approximately 90% of the 15kv to 22 kv output voltage appears above the OV level, thereby providing a polarized signal (DC).

The apparatus for denaturing venoms and toxins 10 also provides a method of treating a plurality of viral conditions such as by way of example but not limitation Herpes simplex—cold sores or fever blisters, Herpes zoster—shingles, and Post Herpetic Neuralgia, Measles, Mumps, even Smallpox, and certain streptomyces and other microbiological organisms. Due to the viral family connections in human and veterinary diseases this method may also be used in treatment of animals. Furthermore, the method may be an alternative to treatment with therapeutic drugs thereby avoiding the associated side effects and costs of therapeutic drugs.

Prior to the advent of antibiotics in the mid 1940-1960s wounds were treated with dyes (allosteric proteins which change shape, depending on the pH, e.g., litmus, brilliant green), and metal ions such as silver. After the initial demonstrated superiority of antibiotics, particularly in systemic bacterial infections, such “old fashioned methods” were abandoned.

It is known that metal ions are generally poisonous to tissue cells. The exceptions to this rule are silver and gold. Silver and gold are poisonous to bacteria but not tissue. This is because in the prokaryote (bacterial) cell there are no subcelluar membranous organelles, or even a membrane around the nucleus. Therefore, the choice of pure silver or gold (more preferably silver) but also a combination thereof as the terminal 52 and silver or gold as the plurality of electrodes 54 to electrostatically treat the wound would be harmless to the wounded tissue, but inimical to any bacteria present.

Although the distribution system 16 is shown and described as a motor 46, a gear box 48, a rotor 50, a terminal 52, and a plurality of electrodes 54, those skilled in the art will readily recognize and appreciate that there are a variety of mechanical, electrical or electronic distribution systems which could be utilized to disperse the output volt over the area of envenomation.

Changes may be made in the construction and the operation of the various components, elements and assembles described herein and changes may be made in the steps or the sequence of steps of the methods described herein without departing from the spirit and scope of the invention as defined in the following claims.

What is claimed is:

1. An apparatus for electrically denaturing venoms and toxins in a patient, comprising:
a voltage generator having a first output and a second output, the voltage generator generating a voltage potential between the first output and the second output; and
a distributor system including a terminal conductively connected to the first output of the voltage generator, and a plurality of electrodes in proximity to the terminal, the terminal and electrodes capable of being disposed in proximity to an area of envenomation on a patient, each of the plurality of electrodes electrically isolated from each other and each of the plurality of electrodes in intermittent conductive contact with the second output of the voltage generator such that the voltage potential is intermittently discharged and distributed between the terminal and each of the plurality of electrodes.

2. The apparatus of claim 1 further including a housing that at least partially encloses the voltage generator and distributor system.

3. The apparatus of claim 1 wherein the voltage generator is a variable voltage generator.

4. The apparatus of claim 2 wherein the voltage generator is a variable voltage generator.

5. The apparatus of claim 1 further including a first switch for activating and charging the apparatus and a second switch for discharging the apparatus.

6. The apparatus of claim 1 wherein the voltage potential generated by the voltage generator is in a range from approximately 15 kilovolts to about 22 kilovolts.

7. The apparatus of claim 6 wherein the amperage levels are in a range from about 13 milliamps to about 18 milliamps.

8. The apparatus of claim 1 wherein the voltage generator includes an oscillator, a stepup transformer, an accumulator, an arc gap switch, and an output transformer.

9. The apparatus of claim 1 wherein the distributor system includes a motor, a gear box and a rotor.

10. The apparatus of claim 10 wherein the distributor system further includes a plurality of electrodes surrounding the terminal.

11. The apparatus of claim 10 wherein the housing is substantially constructed from a group of materials consisting of plastic, fiberglass, wood or combinations and derivations thereof.

12. The apparatus of claim 11 wherein the plurality of electrodes is substantially constructed from silver.

13. The apparatus of claim 11 wherein the plurality of electrodes is substantially constructed from gold.

14. The apparatus of claim 11 wherein the plurality of electrodes is constructed of a combination of gold and silver.
